(19) United States
(12) Patent Application Publication
(10) Pub. No.: US 2017/0014020 A1
(43) Pub. Date: Jan. 19, 2017

(54) Colonoscope and Forward Movement Control Method of a Camera Module of the Colonoscope

(71) Applicant: Chin-Shun Tseng, Kaohsiung City (TW)

(72) Inventor: Chin-Shun Tseng, Kaohsiung City (TW)

(21) Appl. No.: 15/208,683

(22) Filed: Jul. 13, 2016

(30) Foreign Application Priority Data
Jul. 16, 2015 (TW) 104123100

(51) Int. Cl. A61B 1/00; H04N 5/225; A61B 1/04; A61B 1/018; A61B 1/045; A61B 1/015; A61B 1/05; A61B 5/06; H04N 5/232; A61B 1/31

(52) U.S. Cl. CPC A61B 1/00156; H04N 5/232; H04N 5/2256; A61B 1/041; A61B 1/31; A61B 1/045; A61B 1/00029; A61B 1/015; A61B 1/051; A61B 5/062; A61B 5/067; A61B 1/00158; A61B 1/00114; A61B 1/00016; A61B 1/00032; A61B 1/018; A61B 1/00006

(57) ABSTRACT

A colonoscope having an egg-shaped camera module and a wiring unit, as well as a forward movement control method of the colonoscope, are disclosed. The camera module includes a casing, a first camera unit, a vibration motor and a control unit. The casing has first and second ends. The first end is made of a transparent material. The first camera unit captures a first image. The vibration motor is adapted to vibrate the casing. The control unit controls the first camera unit to capture the first image. The control unit controls the vibration of the vibration motor. The wiring unit forms a vent and includes at least two lead wires and an air tube. Power can be transmitted to the control unit through the lead wires. The air tube is adapted to convey air, and the vent is adapted to output the air to a colon.

### CROSS REFERENCE TO RELATED APPLICATIONS

The application claims the benefit of Taiwan application serial No. 104123100, filed on Jul. 16, 2015, and the subject matter of which is incorporated herein by reference.

### BACKGROUND OF THE INVENTION

1. Field of the Invention

The present disclosure generally relates to a colonoscope and, more particularly, to a colonoscope having an egg-shaped camera module and a forward movement control method of the egg-shaped camera module of the colonoscope.

2. Description of the Related Art

In Taiwan, cancers have been the leading cause of death for 33 consecutive years. Among various cancers, colon cancer and rectal cancer have remained the top three causes of death. In 2014, there were up to 15,000 people who were diagnosed with cancers, which resulted in a death toll of 5,000. In Japan, there are 45,000 people who are diagnosed with cancers every year, which resulted in a death toll of 18,000. In America, there are 150,000 people who are diagnosed with cancers every year, which leads to a death toll of 50,000. The cancers are highly related to the colon polyps. In Taiwan, there are about 710,000 people who have colon polyps among adults aged 20-49 years. The colon polyps may lead to colon cancer in 5-10 years. Thus, if the colon polyps are properly handled, the colon cancer can be prevented in most of the cases.

Currently, colonoscope is the medical instrument that is used to examine the colon polyps. The main manufacturer of the colonoscope is Olympus Medical Systems Corp. Their colonoscopes are constructed by fibers. However, the use of fiber material makes it difficult to bend the tube when it is needed to adjust the position of the camera lens. Secondly, the colonoscope has a length of about 180 cm. Therefore, if it is needed to examine the entire colon in a full length, general anesthetics is required. However, since an anesthetics has some risk, most of the patients are not willing to accept the general anesthetics. In addition, pumping the air into the intestinal tract during the examination will cause abdominal distension, and the insertion and movement of the thick, long tube in the colon will exert a pressing force on the intestinal wall of the colon. Therefore, most of the patients suffer a great pain during the examination, and even have a hard time finishing the colonoscopy. Furthermore, several domestic researches revealed that there is about 0.25% to 0.5% chance that the intestinal perforation may be resulted during the colonoscopy due to some factors such as improper use of the colonoscope, the special condition of the patient, or the hot biopsy of the polyps. This often leads to a medical litigation. Thus, the colonoscopy has a lot of risks itself. In the position of the patients, they are concerned about the risk of intestinal perforation. In the position of the doctors, they are concerned about the risk of medical litigation. Due to the reasons, colonoscopy is not widely accepted, and many people do not know they have colon polyps since they are not willing to do the colonoscopy. This leads to a higher chance of colon cancer.

In light of the deficiency where it is difficult to bend the tube to adjust the movement direction of the tip of the tube, some aids have been proposed to straighten the colon such as an external straighter as disclosed in European Patent No. 0792130B1. Such an aid has a protuberance. Due to this, the aid can press the colon and therefore straighten the colon. Then, the tube can be inserted deep into the colon. A similar aid was also proposed by U.S. Pat. No. 2014/0350341A1. However, these aids are no longer helpful at the sharp bend of the intestinal tract, such as at the location where the descending colon connects to the transverse colon, as well as the location where the transverse colon connects to the ascending colon.

In addition to straightening the colon through the use of the aids, a bendable colonoscope was also proposed (by changing the structure of the colonoscope) to overcome the difficulty in bending the tube of the conventional colonoscope. For example, Taiwan Patent No. I468140 discloses a magnetically-controlled system applicable for colonoscopy. Such a system includes an external magnetic member and an internal magnetic member. The internal magnetic member is mounted on a bar-shaped instrument of the colonoscope, and the external magnetic member is mounted on an external device. Based on this, the external magnetic member can guide the internal magnetic member to change its movement direction. As such, the bar-shaped instrument is able to change its movement direction at the bends of the colon. Although this type of colonoscope overcomes the difficulty in bending the tube of the conventional colonoscope, its bar-shaped instrument is still as thick as the tube of the conventional colonoscope. Although the bar-shaped instrument of the colonoscope can change its movement direction in the colon, it tends to exert a pressing force on the intestinal wall of the colon if a slight mistake is made (due to poor skill or oversight) during the movement of the bar-shaped instrument, resulting in a hard contact between the bar-shaped instrument and the intestinal wall of the colon. As a result, the patients still suffer a great pain during the examination.

Besides, for any type of the conventional colonoscope (including the one disclosed in Taiwan Patent No. I468140), there exists some blind spots around the folds of the colon where the viewing thereof is hardly possible. In other words, the conventional colonoscope has only one direction of view. Disadvantageously, the colonoscope is not able to view the back side of the fold, leading to an incomplete examination.

Therefore, a colonoscope that provides a nearly pain-free examination will certainly be advantageous in preventing the colon cancer. It has been an important issue among the manufacturers of the medical instrument to develop a colonoscope which is easy to operate, has no blind spot, provides a nearly pain-free examination, and meets various demands of the doctors.

### SUMMARY OF THE INVENTION

It is therefore an objective of the disclosure to provide an egg-shaped camera module of a colonoscope, in which the colonoscope has a highly flexible tube, is easy to operate, and provides a nearly pain-free colonoscopy.

In an embodiment of the disclosure, a colonoscope having an egg-shaped camera module, a wiring unit and a power supply member is disclosed. The camera module includes a casing, a first camera unit, a vibration motor and a control unit. The casing has a first end and a second end. The first and second ends are spaced from each other in an axial direction thereof. The first end is made of a transparent material. The first camera unit is arranged at the first end of the casing to provide an illumination effect and to capture a first image in a first direction. The vibration motor is arranged in the casing to vibrate the casing. The control unit is arranged in the casing and electrically connected to the first camera unit and the vibration motor. The control unit controls the first camera unit to capture the first image and controls the transmission of the captured first image upon the reception of a command. The control unit controls the vibration of the vibration motor. The wiring unit is fixed to the second end of the casing and includes an outer sheath and an air tube. The air tube is made of a soft material and is enveloped in the outer sheath. The power supply member is disposed in the casing or the wiring unit and is electrically connected to the control unit. The power supply member provides power to the control unit. The casing or the wiring unit forms a vent that is in communication with the air tube of the wiring unit. The air tube is adapted to convey air, and the vent is adapted to output the air to a colon.

In another embodiment of the disclosure, a forward movement control method of a camera module of a colonoscope is disclosed. The camera module includes a vibration motor, a wiring unit, a first end, a second end and a first camera unit. The vibration motor is used to vibrate the casing. The wiring unit is used to transmit power. The first and second ends are spaced from each other in an axial direction of the casing. The first camera unit is located at the first end of the casing. The method includes vibrating the camera module, and slanting the camera module in an inclined state where the first end of the casing is in a lower level than the second end is.

In a further embodiment of the disclosure, a forward movement control method of a camera module of a colonoscope is disclosed. The camera module includes a wiring unit, a first end, a second end, a first camera unit and a telescopic movement control unit. The wiring unit is used to transmit power. The first and second ends are spaced from each other in an axial direction of the camera module. The first camera unit is located at the first end of the casing. The telescopic movement control unit is used to control the camera module to move telescopically along the axial direction of the camera module. The method includes moving the camera module telescopically, and slanting the camera module in an inclined state where the first end of the camera module is in a lower level than the second end is.

### BRIEF DESCRIPTION OF THE DRAWINGS

The present disclosure will become more fully understood from the detailed description given hereinafter and the accompanying drawings which are given by way of illustration only, and thus are not limitative of the present disclosure, and wherein:

FIG. 1A is a cross sectional view of an egg-shaped camera module of a colonoscope according to a first embodiment of the disclosure.

FIG. 1B is a cross sectional view of the egg-shaped camera module of the colonoscope of the first embodiment where the vibration motor is mounted to a different position of the central cylinder of the casing.

FIG. 2 is a cross sectional view of the egg-shaped camera module of the colonoscope of the first embodiment where the casing forms a vent.

FIG. 3A is a cross sectional view of the egg-shaped camera module of the colonoscope of the first embodiment where the casing is provided with a plurality of movement auxiliaries.

FIG. 3B shows the egg-shaped camera of FIG. 3A where each of the plurality of movement auxiliaries is in an annular form.

FIG. 3C shows the egg-shaped camera of FIG. 3A where each of the plurality of movement auxiliaries is in a helical form.

FIG. 4A is a cross sectional view of the egg-shaped camera module of the first embodiment, in which the camera module is provided with the at least one internal magnetic member.

FIG. 4B is a radially cross sectional view of the camera module of FIG. 4A.

FIG. 5 is a cross sectional view of the egg-shaped camera module wherein a wireless transmission module is provided.

FIG. 6 shows a use of the egg-shaped camera module in a colon according to the first embodiment of the disclosure.

FIG. 7A shows the egg-shaped camera module which is located in a position where a polyp cannot be viewed.

FIG. 7B shows the egg-shaped camera module which moves to another position where the polyp can be viewed.

FIG. 8A shows a flowchart of a forward movement control method of the egg-shaped camera module according to a second embodiment of the disclosure.

FIG. 8B shows a flowchart of a forward movement control method of the egg-shaped camera module according to a third embodiment of the disclosure.

FIG. 9A is a cross sectional view of an egg-shaped camera module of a colonoscope according to a fourth embodiment of the disclosure.

FIG. 9B is a cross sectional view of an exposed part of a wiring unit of the camera module shown in FIG. 9A.

FIG. 9C is a radially cross sectional view of the first end of the casing shown in FIG. 9A.

FIG. 10 is a cross sectional view of the egg-shaped camera module of FIG. 9A where a polypectomy snare is used in the surgery.

FIG. 11A is a cross sectional view of an egg-shaped camera module of a colonoscope according to a fifth embodiment of the disclosure.

FIG. 11B is a cross sectional view of an exposed part of a wiring unit of the camera module of FIG. 11A.

FIG. 11C is a cross sectional view of the first end of the camera module of FIG. 11A.

FIG. 12 is a cross sectional view of the egg-shaped camera module of FIG. 11A where a polypectomy snare is used in the surgery.

FIG. 13A is a cross sectional view of an egg-shaped camera module of a colonoscope according to a sixth embodiment of the disclosure where the casing of the camera module is in a fully-stretched state.

FIG. 13B is a cross sectional view of the egg-shaped camera module of FIG. 13A where the casing of the camera module is in a fully-retracted state.

FIG. 13C is a cross sectional view of the egg-shaped camera module of FIG. 13A where the casing of the camera module is partially stretched.

In the various figures of the drawings, the same numerals designate the same or similar parts. Furthermore, when the terms “first”, “second”, “third”, “fourth”, “inner”, “outer”, “top”, “bottom”, “front”, “rear” and similar terms are used hereinafter, it should be understood that these terms have reference only to the structure shown in the drawings as it would appear to a person viewing the drawings, and are utilized only to facilitate describing the disclosure.

### DETAILED DESCRIPTION OF THE INVENTION

In an embodiment of the disclosure, a colonoscope having an egg-shaped camera module and a forward movement control method of the camera module are disclosed. The camera module can be smoothly contained in the intestinal tract of a patient due to its egg shape. Then, the camera module can move along the intestinal tract under a vibration force. Although the camera module needs to change its movement direction at the bends of the intestinal tract, it will not cause uncomfortable feeling of the patient. The camera module is also connected with a soft wiring unit which provides the required power to the camera module. Air (or gas) can also be pumped into the intestinal tract to properly expand the intestinal tract under the operation of the doctor, allowing the doctor to examine the condition of the colon. The detailed structure and function of the camera module is discussed below.

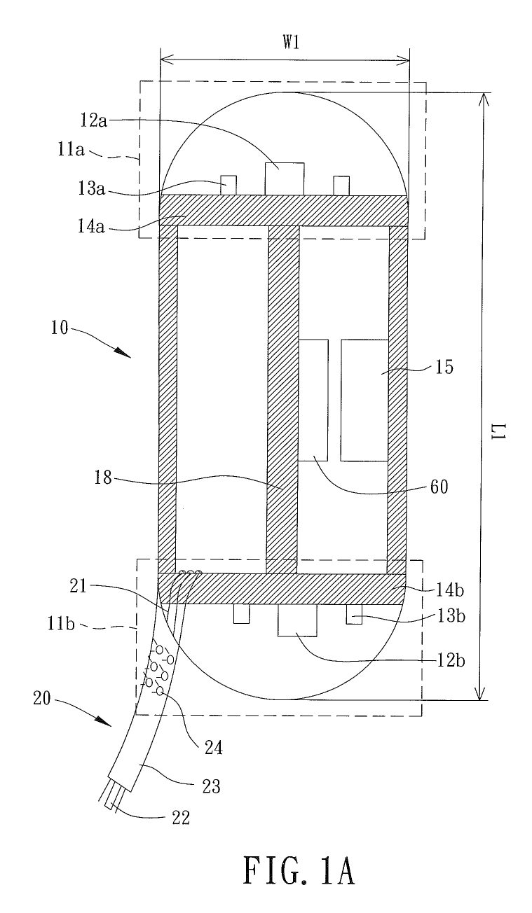

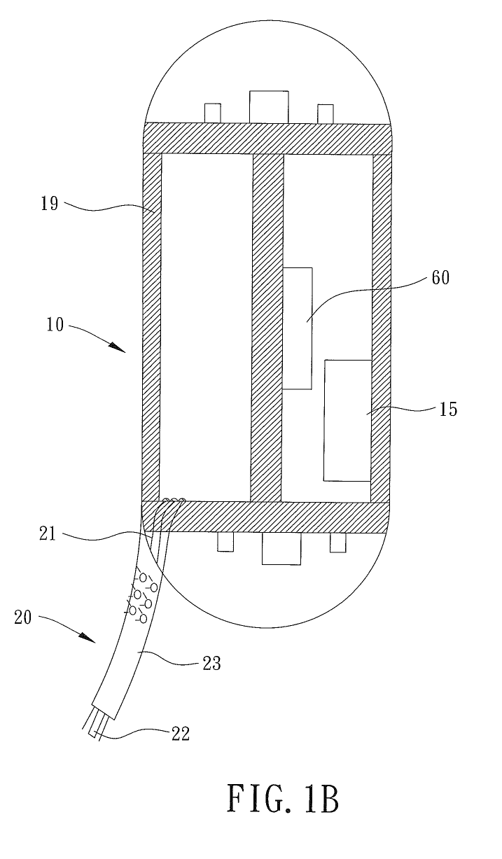

FIG. 1A is a cross sectional view of an egg-shaped camera module of a colonoscope according to a first embodiment of the disclosure. The camera module includes an egg-shaped casing 10 and a wiring unit 20. The egg-shaped casing 10 includes a first camera unit, a second camera unit, a vibration motor 15 and a control unit 60. The egg-shaped casing 10 has a first end 11a, a second end 11b and a central cylinder. The first end 11a and the second end 11b are opposite to each other in an axial direction of the camera module. The central cylinder extends in the axial direction of the camera module. Both the first end 11a and the second end 11b are made of a transparent material. The first camera unit is arranged at the first end 11a, and includes a first image capturing device 12a, a first light emitting unit 13a and a first circuit board 14a. The second camera unit is arranged at the second end 11b, and includes a second image capturing device 12b, a second light emitting module 13b and a second circuit board 14a. The first and second first camera units are used to provide an illumination effect and capture the images (such as video image). The first camera unit is used to capture the image in a first direction, and the second camera unit is used to capture the image in a second direction substantially opposite to the first direction. When the first camera unit captures the image in the first direction, there are always some blind spots around the folds of the colon which are hidden from the first camera unit. In light of this deficiency, the second camera unit is used to capture the images of the blind spots of the first camera unit. The vibration motor 15 is arranged in the egg-shaped casing 10 and provides a vibration force for the casing 10. The control unit 60 is arranged in the egg-shaped casing 10. In the first embodiment, the control unit 60 is arranged on a central circuit board 18. The central circuit board 18 is connected to the first circuit board 14a and the second circuit board 14b via a wire system. However, this is not used to limit the scope of the disclosure. The control unit 60 is connected to the first camera unit, the second camera unit and the vibration motor 15. Upon the reception of an external command, the control unit 60 may control the first and second camera units to capture the images and controls the transmission of the captured images. Based on this, the control unit 60 can control the vibration motor 15 to vibrate or not. The wiring unit 20 is fixed to the second end 11b of the casing 10, and includes at least two lead wires 21 and an air tube 22 that are enveloped in an outer sheath 23. In the first embodiment, the at least two lead wires 21 are shown to include three lead wires, which are a positive power line, a negative power line and a signal line. Also, the outer sheath 23 in the first embodiment includes a plurality of vents 24. The at least two lead wires 21 serve as a power supply member electrically connected to the control unit 60. Electricity can be supplied to the elements in the casing 10 (such as the first and second camera units, the control unit 60 and the vibration motor 15) through the power supply member. The pumped air can be outputted into the colon via the plurality of vents 24.

In the first embodiment, the vibration motor 15 is mounted to an inner wall 19 of the casing 10. Namely, the vibration motor 15 is mounted to an inner wall of the central cylinder of the casing 10. However, according to a first modification of the first embodiment, the vibration motor 15 can also be mounted to the central circuit board 18. Besides, the vibration motor 15 is mounted to a central position of the central cylinder of the casing 10 as shown in FIG. 1A. However, the vibration motor 15 can also be mounted to a lower position of the central cylinder of the casing 10 which is adjacent to the second end 11b as shown in FIG. 1B, or to an upper position of the central cylinder of the casing 10 which is adjacent to the first end 11a. In the cases of lower and upper positions, the upper position is preferred.

Both the first and second image capturing devices 12a and 12b may include a high-resolution CMOS or CCD sensor. Both the first and second light emitting units 13a and 13b may include a light-emitting module formed by light-emitting diodes (LED).

The casing 10 may have a length L1 between 2.5 and 5.2 cm and a width W1 between 1.5 and 2.5 cm.

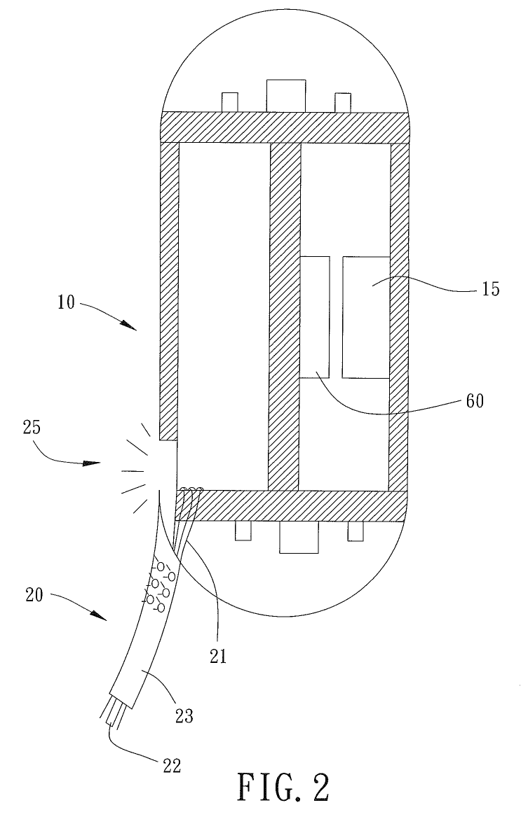

The action of pumping air into the intestinal tract is an external operation, but this is the main function that the colonoscope needs to provide. Although a plurality of vents 24 can be formed on the wiring unit 20 at a location adjacent to the second end 11b of the casing 10 as shown in FIG. 1A, the casing 10 may also form a vent 25 in replacement of the plurality of vents 24 as shown in FIG. 2.

Although the air can be pumped into the intestinal tract via the vent 25, water or medicinal liquid may also be pumped into the intestinal tract according to the requirement.

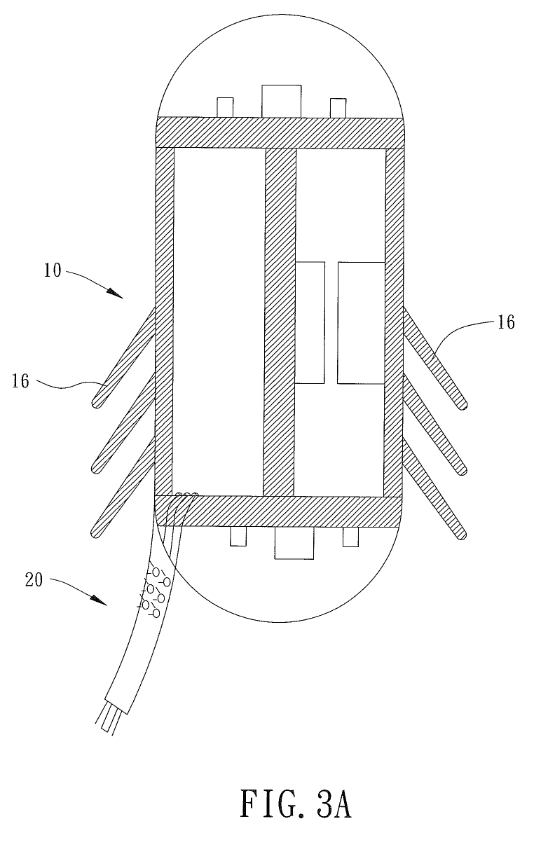

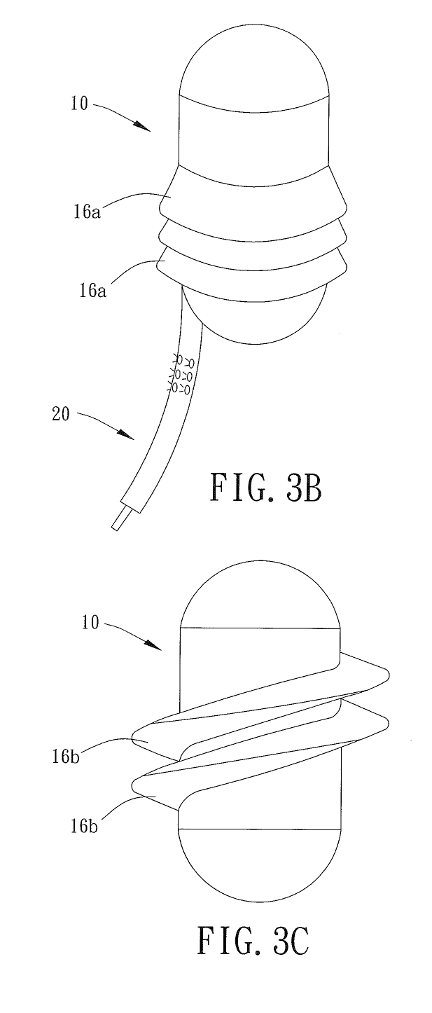

To facilitate the movement of the camera module, the casing 10 of the camera module can be provided with at least one movement auxiliary 16 (each may be in the form of a protrusion formed on the casing 10). As shown in FIGS. 3A, 3B and 3C, the at least one movement auxiliary 16 may include a first movement auxiliary 16a and a second movement auxiliary 16b. The arrangement of the first and second movement auxiliaries 16a and 16b ensures a smooth movement of the casing 10 in the colon. Each of the first and second movement auxiliaries 16a and 16b may be in an annular form (as shown in FIG. 3B), a helical form (as shown in FIG. 3C) or in the shape of snake's scales, or any combination thereof.

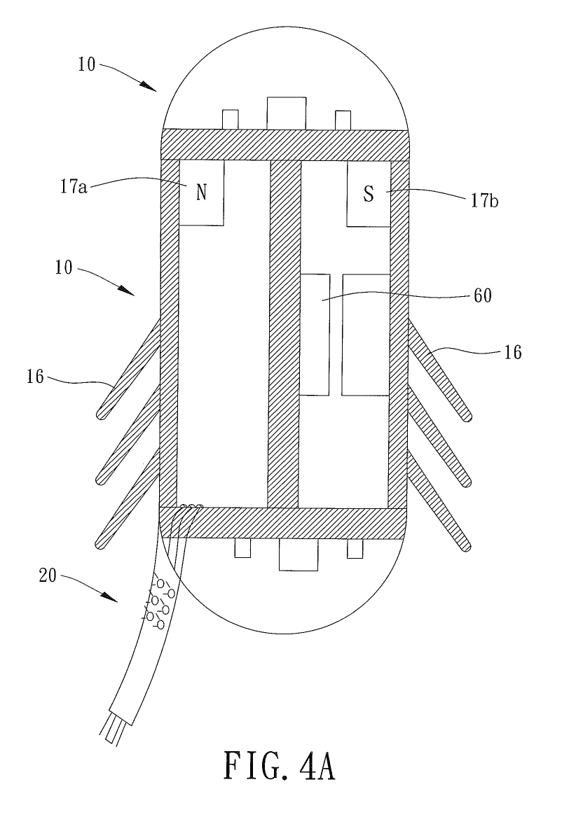

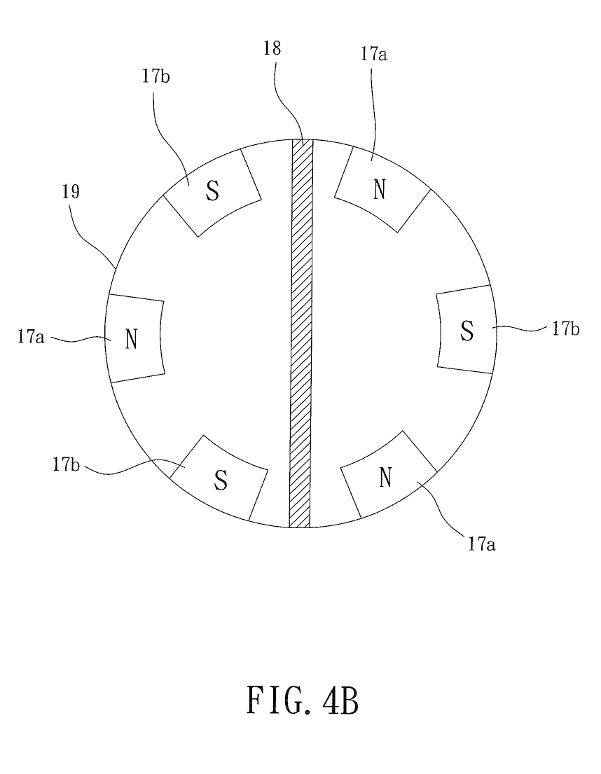

In addition to the at least one movement auxiliary 16, the egg-shaped camera module may also include at least one internal magnetic member according to a second modification of the first embodiment of the disclosure. FIG. 4A is a cross sectional view of the egg-shaped camera module that is provided with the at least one internal magnetic member, and FIG. 4B is a top view of the camera module. In the second modification of the first embodiment of the disclosure, three internal magnetic members are arranged on the central cylinder at a position adjacent to the first end 11a. Each of the internal magnetic members includes an N-pole element 17a and an S-pole element 17b. Based on this, an external magnetic member that is magnetically attracted to the internal magnetic member can be provided. The external magnetic member can be used to control the positioning and movement of the casing 10 through the magnetic force between the internal and external magnetic members. Since some parts of the intestinal wall may be hidden from the second camera unit due to the obstruction of the wiring unit 20, the casing 10 can be turned by an angle (about a vertical axis) to adjust the viewing angle of the second camera unit. Specifically, the doctor can approach the external magnetic member to the internal magnetic member. When the external and internal magnetic members are close enough to each other, they will be magnetically attracted to each other by the magnetic force. In this regard, when the doctor turns the external magnetic member by an angle, the internal magnetic member also turns by the angle. As a result, the casing 10 is turned and the viewing direction of the second camera unit is changed. In another case, the doctor can move the external magnetic member forward instead of turning the external magnetic member. In this situation, the moved external magnetic member will bring the internal magnetic member to move forward, thus facilitating the forward movement of the casing 10. Furthermore, the precise location of the casing 10 in the intestinal tract can be detected through the magnetic field generated by the internal magnetic member, allowing the doctor to precisely indicate the location of the affected part.

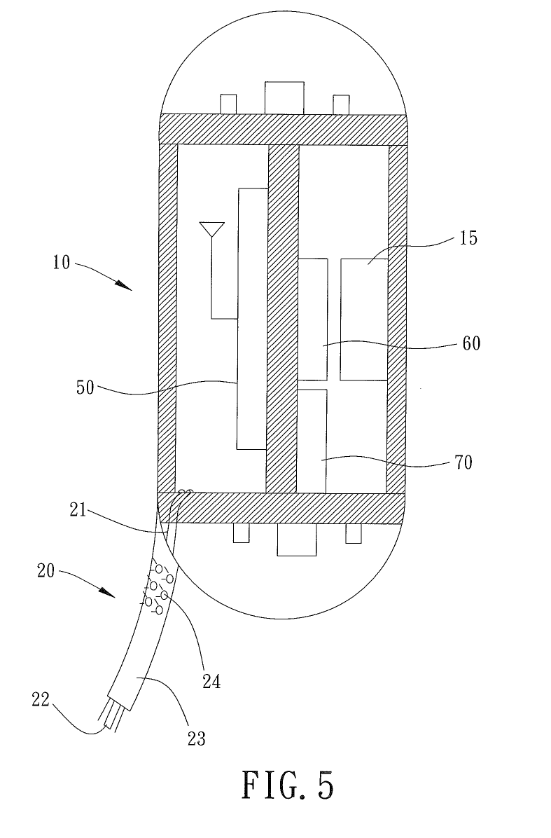

Moreover, due to the arrangement of the wiring unit 20, there will be a sufficient amount of power for image transmission. The image can be transmitted to an outer device in a wired or wireless manner. In the wired transmission, signals can be transmitted over the signal line or the power line. Therefore, the wiring unit 20 may include a signal line. Alternatively, the casing 10 may be further provided with a power-line signal transmission module. The control unit 60 can transmit the instant images of the first and second camera units to the outer device in either case. In addition, FIG. 5 shows a third modification of the first embodiment of the disclosure. In FIG. 5, a wireless transmission module 50 is provided. The wireless transmission module 50 is connected to the control unit 60 and is used to transmit the images of the first and second camera units to the outer device.

Besides, in the case of wireless transmission, a battery can be provided in the casing 10 to replace the wiring unit 20. The battery can provide the required power of the casing 10, therefore the at least two lead wires 21 can be omitted. The wireless transmission module 50 not only transmits the images captured by the first and second camera units, but also receives and transmits an external command to the control unit 60. Thus, the wiring unit 20 does not need to include the at least two lead wires 21. The wiring unit 20 only needs to include the air tube 22 to perform the function of the egg-shaped camera module of the disclosure.

FIG. 5 shows a fourth modification of the egg-shaped camera module of the first embodiment of the disclosure. In the fourth modification, the egg-shaped camera module further includes an angle detection unit 70 connected to the control unit 60. The angle detection unit 70 is used to detect the angle of the casing 10 with respect to the horizontal line. The detected result can be transmitted to the outer device by the control unit 60. The angle detection unit 70 is used to detect the inclined angle of the egg-shaped camera module when the camera moves in the intestinal tract. Namely, the angle detection unit 70 can detect whether the egg-shaped camera module is in an inclined state where the first end 11a of the casing 10 is heading downwards (i.e. the first end 11a of the casing 10 is in a lower level than the second end 11b is). If the egg-shaped camera module is in the inclined state, the camera module can move in the colon under a vibration force. If the egg-shaped camera module is not in an inclined state (the first end 11a of the casing 10 is in a higher level than the second end 11b or is in the same level as the second end 11b), the doctor can guide the egg-shaped camera module to move along the intestinal tract using the external magnetic member shown in FIG. 4. In another option, the doctor can adjust the lying posture of the patient to change the inclined angle of the intestinal tract, so as to place the egg-shaped camera module in an inclined state. Therefore, arrangement of the angle detection unit 70 can facilitate the forward movement of the egg-shaped camera module.

The angle detection unit 70 may be a microelectromechanical angle detection chip, a microelectromechanical gyroscope chip, a microelectromechanical dual-axis acceleration detection chip, a microelectromechanical tri-axis acceleration detection chip, a rolling switch, or a magnetic sensor. Selection of the above elements may be based on space occupation and vibration resistance. The one with smaller volume and higher vibration resistance is preferred.

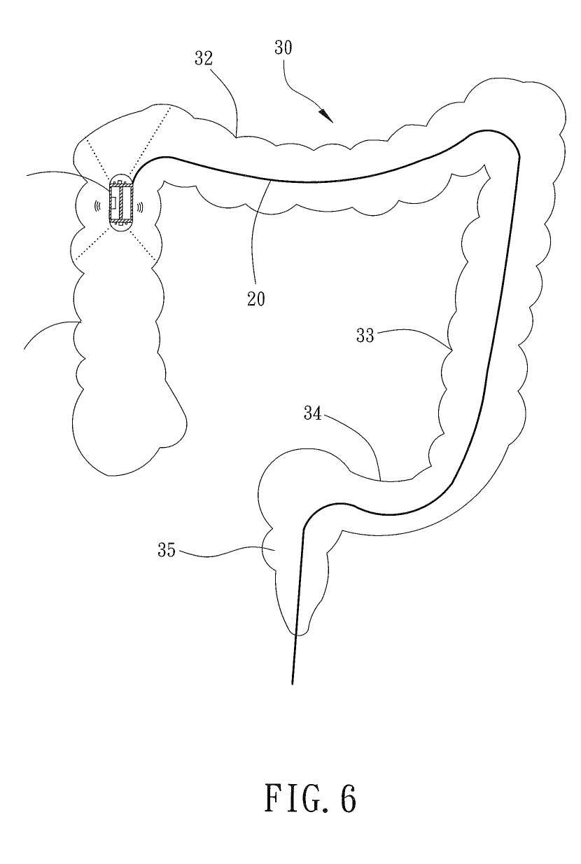

FIG. 6 shows a use of the egg-shaped camera module in a colon according to the first embodiment of the disclosure. It can be seen from FIG. 6 that the camera module has an egg shape that fits to the intestinal tract and can be smoothly contained therein. Moreover, since the wiring unit 20 uses an air tube formed by a soft material, the air tube is highly flexible and has a diameter smaller than 0.5 cm. Therefore, the wiring unit 20 does not exert a pressing force on the intestinal wall of the colon. As a great advantage, the patient will not feel uncomfortable when the egg-shaped camera module moves in the intestinal tract of the patient. Thus, the egg-shaped camera module can move through the entire intestinal tract (in a full length of 160 cm, including the rectum 33, the sigmoid 34, the descending colon 33, the transverse colon 32 and the ascending colon 31) in a nearly pain-free manner.

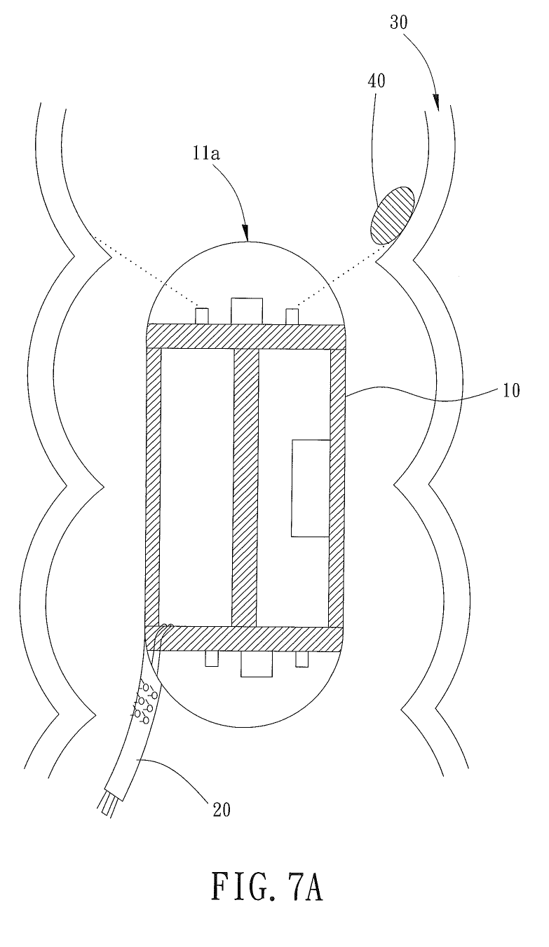

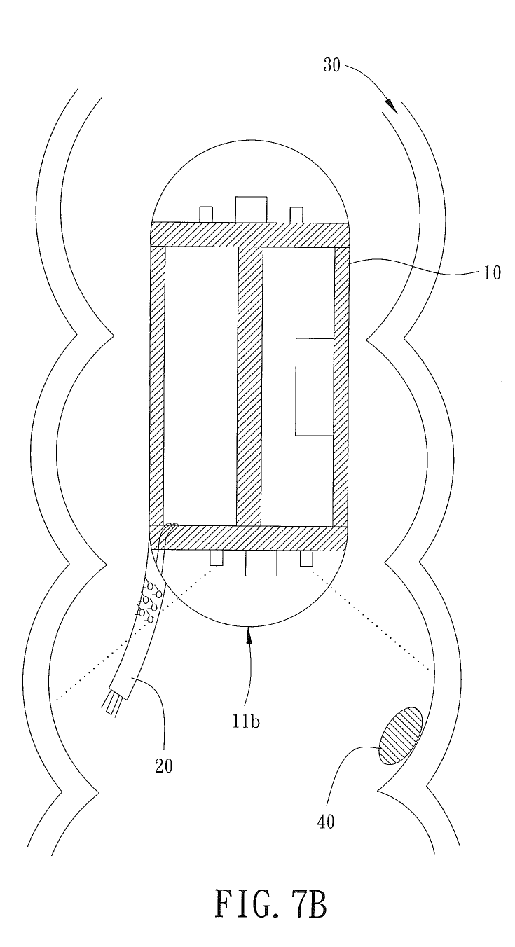

More importantly, the dual-camera design of the egg-shaped camera module of the first embodiment of the disclosure is able to provide a full-angle viewing of the internal structure of the colon without any blind spot. As such, it is possible to view the back side of the fold. FIGS. 7A and 7B show a use of the egg-shaped camera module. In FIG. 7A, when the egg-shaped camera module moves to a position in the colon 30, a polyp 40 may be located at a back side of the fold where the first camera unit is not able to view. However, as the egg-shaped camera module continuous to move to another position as shown in FIG. 7B, the second camera unit is able to view the polyp 40 which the first camera unit was unable to view.

In the disclosure, the egg-shaped camera module may be driven by the vibration force to move forward. The use of vibration force as a power source can reduce the uncomfortable feeling of the patient. Thus, the forward movement control method of the camera module is critical to achieving the desired advantage of the disclosure. There are three approaches to drive the egg-shaped camera module of the first embodiment of the disclosure. In the first approach, the egg-shaped camera module can move under the gravitational force and the vibration force. In the second approach, the egg-shaped camera module can move under the magnetic force and the vibration force. In the third approach, the egg-shaped camera module can move under the gravitational force, the magnetic force and the vibration force altogether.

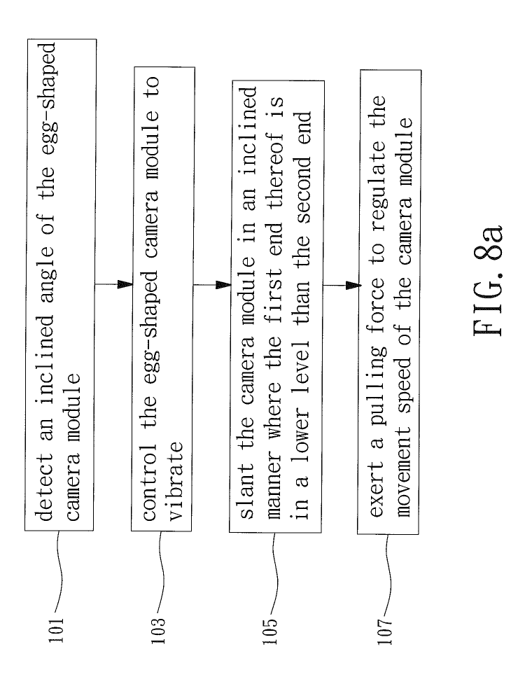

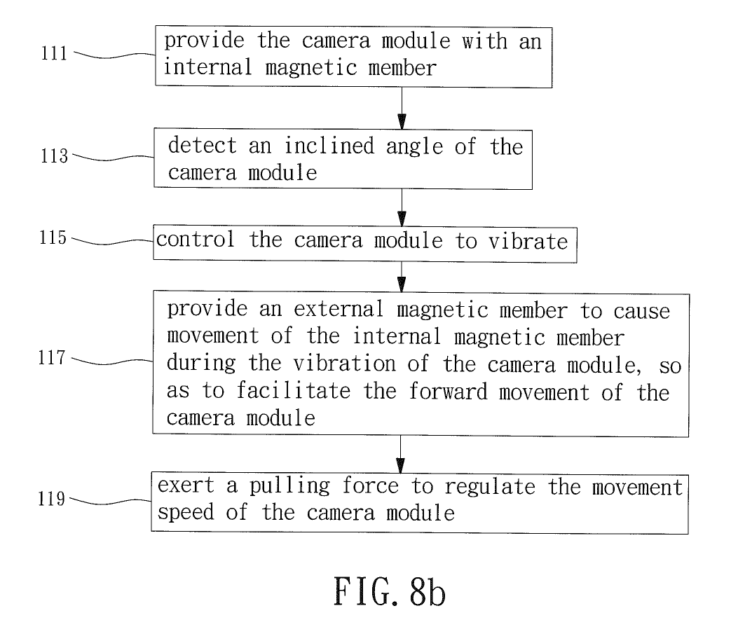

FIG. 8A shows a flowchart of a forward movement control method of the egg-shaped camera module according to a second embodiment of the disclosure, which includes steps 101, 103 and 105, as elaborated below.

In the step 101, an inclined angle of the egg-shaped camera module is detected. In the step 103, the egg-shaped camera module is controlled to vibrate. In the step 105, during the vibration, the egg-shaped camera module is slanted in an inclined manner where the first end 11a thereof is in a lower level than the second end 11b. The egg-shaped camera module can be slanted by deviating the intestinal tract from the horizontal line. In this case, the egg-shaped camera module is heading downwards. Specifically, since the inclined angle of the intestinal tract is detected and the inner condition of the intestinal tract is observed by the first camera unit, there are many ways to adjust the inclined angle of the intestinal tract if the egg-shaped camera module is not in the desired inclined state (with the first end 11a not heading downwards). In one of the approaches, the doctor can adjust the lying gesture of the patient to slant the intestinal tract in a desired inclined manner. In another approach, the doctor can incline the sickbed to slant the intestinal tract of the patient in a desired inclination. More specifically, when the egg-shaped camera module reaches the descending colon, the doctor can incline the sickbed to lift the head of the patient and to lower the legs of the patient. In this situation, the egg-shaped camera module can be in an inclined state where the first end 11a thereof is heading downwards. Then, based on the inclined angle of the intestinal tract, the doctor can adjust the local position of the intestinal tract in order to slant the egg-shaped camera module in the desired inclined state. As such, the first end 11a of the egg-shaped camera module can be heading downwards, allowing the camera module to move along the descending colon more easily. As another example, when the egg-shaped camera module reaches the bend between the descending colon and the transverse colon, the patient can be in the right side lying position to allow the camera module to move more easily in the transverse colon. Alternatively, the doctor can squeeze a part of the intestinal tract to allow the egg-shaped camera module to move more smoothly. In addition to the steps 101, 103 and 105, the forward movement control method of the egg-shaped camera module according to the second embodiment of the disclosure may further include a step 107. In the step 107, a pulling force is exerted to regulate the movement speed of the egg-shaped camera module and to adjust the movement direction of the camera module. The step 107 is provided to slow down the egg-shaped camera module when the camera module moves too fast, as well as to adjust the direction of the camera module when the camera module deviates from the desired movement direction and gets stuck in the intestinal tract. The pulling force is exerted by pulling the wiring unit 20.

FIG. 8B shows a flowchart of a forward movement control method of the egg-shaped camera module according to a third embodiment of the disclosure. The forward movement control method in the third embodiment uses magnetic force as a power source of the egg-shaped camera module. Namely, the forward movement control method uses magnetic force to control the movement of the egg-shaped camera module shown in FIG. 4A. The flowchart includes steps 111, 113, 115 and 117, as elaborated below.

In the step 111, an internal magnetic member is provided. In the step 113, an inclined angle of the egg-shaped camera module is detected. In the step 115, the egg-shaped camera module is controlled to vibrate. In the step 117, an external magnetic member is provided to cause movement of the internal magnetic member during the vibration, so as to facilitate the forward movement of the egg-shaped camera module. Since the inclined angle of the egg-shaped camera module is detected, the external magnetic member can be used to guide the camera module to move forward during the movement thereof. This can be observed from the image captured by the first camera unit. Thus, during the vibration of the egg-shaped camera module, the external magnetic member can provide a small auxiliary momentum to facilitate the forward movement of the camera module.

Similarly, in addition to the steps 111, 113, 115 and 117, the forward movement control method of the egg-shaped camera module according to the third embodiment of the disclosure may further include a step 119. In the step 119, a pulling force is exerted to regulate the movement speed of the egg-shaped camera module and to adjust the movement direction of the camera module. The step 119 is not elaborated herein as it is similar to the step 107 previously discussed.

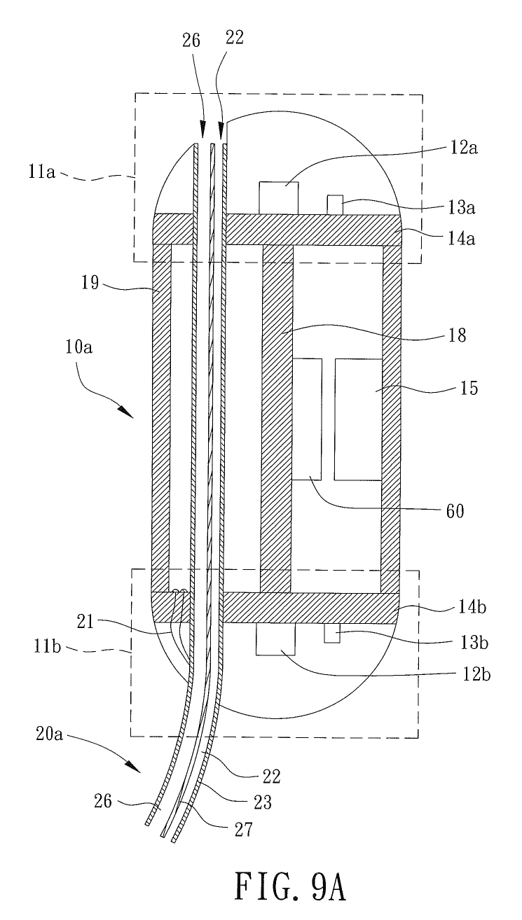

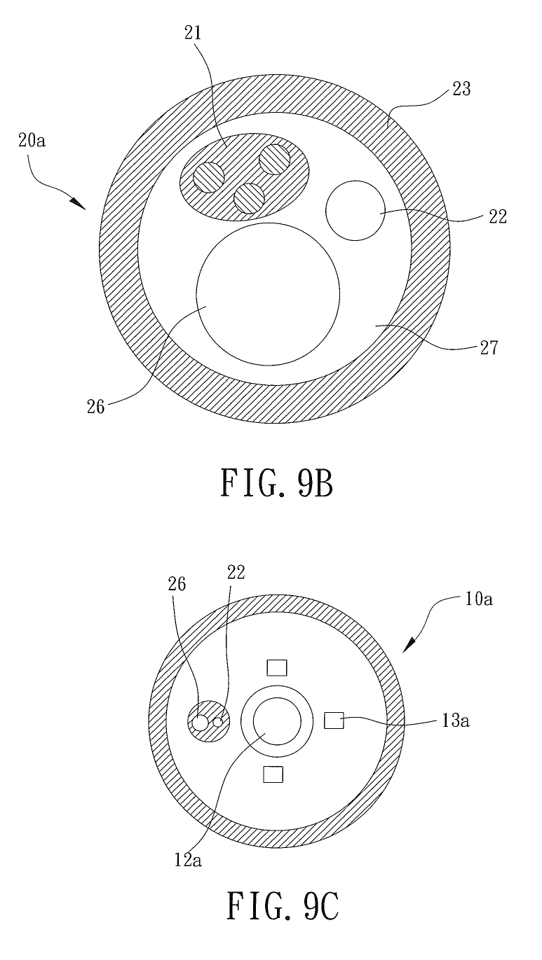

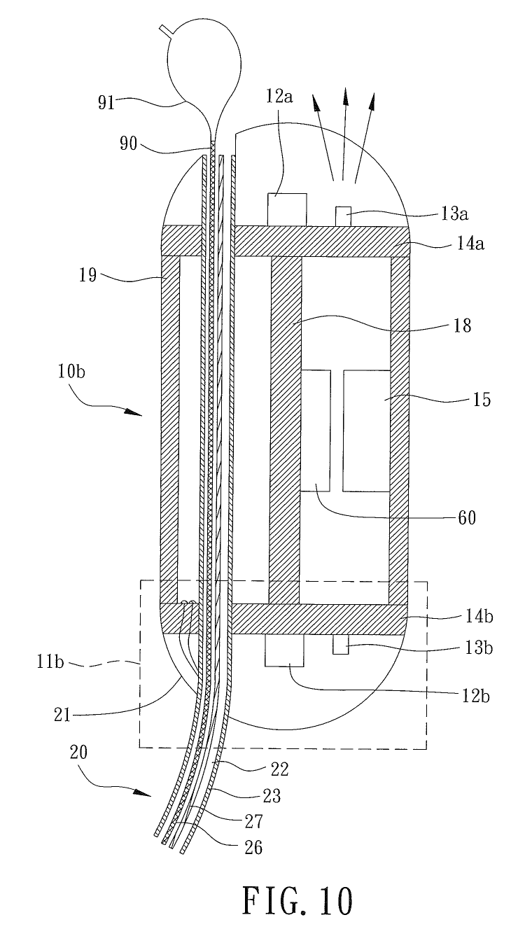

FIG. 9A is a cross sectional view of an egg-shaped camera module of a colonoscope according to a fourth embodiment of the disclosure. FIG. 9B is a cross sectional view of an exposed part of a wiring unit of the camera module shown in FIG. 9A. FIG. 9C is a cross sectional view of the first end 11a of the casing 10 shown in FIG. 9A. In the fourth embodiment, the wiring unit 20a further includes an instrument channel 26 in addition to the air tube 22. The air tube 22 and the instrument channel 26 lead to the same outlet at the first end 11a of the casing 10. In the fourth embodiment, the outlet is arranged near the first image capturing device 12a, so that a polypectomy snare 90 (as shown in FIG. 10), a hemostatic needle, a biopsy forcep or a foreign body forcep can be extended into the instrument channel 26 to perform the related surgery. The egg-shaped camera module of the colonoscope according to the fourth embodiment of the disclosure can also include the internal and external magnetic members as shown in FIGS. 4A and 4B. As such, the internal and external magnetic members can be magnetically attracted to each other under the magnetic force during the surgery, so that the doctor is able to perform the related surgery on the affected part.

In the cross sectional view of the wiring unit 20a as shown in FIG. 9B, the wiring unit 20a further includes the instrument channel 26 and a filling 27 in addition to the lead wires 21 (including the positive power line, the negative power line, the signal line and the envelope) and the air tube 22. The filling 27 can provide a reinforced effect for the wiring unit 20a. In FIG. 9A, the outlet at the first end 11a does not include the lead wires 21. Instead, the lead wires 21 extend out of the outer sheath 23 at the second end 11b and are welded to the second circuit board 14b. This forms a special type of the wiring unit 20a. However, the lead wires 21 may also extend out of the outer sheath 23 at the first end 11a and are welded to the first circuit board 14a (this is not shown).

In FIG. 9C, the air tube 22 and the instrument channel 26 are next to the first image capturing device 12a. As such, when an instrument extends out of the instrument channel 26, the doctor can clearly see the instrument and accurately perform the surgery as shown in FIG. 10.

FIG. 10 is a cross sectional view of the egg-shaped camera module of FIG. 9A where the polypectomy snare 90 is extended through the camera module in order to perform the surgery. In this arrangement, air can be pumped into the intestinal tract through the air tube 22 to expand the colon, and liquid can be sprayed into the intestinal tract through the air tube 22 to lubricate the intestinal tract. The first light emitting unit 13a can be controlled to generate different colors of light. As such, a respective color of light can be irradiated into the intestinal tract, and the first image capturing device 12a can capture the images under the illumination of different colors of light. Alternatively, the first image capturing device 12a can be replaced by a lens with adjustable zoom ratio so that the first image capturing device 12a can capture the image in a low or high zoom ratio. The first image capturing device 12a can capture the image of the cells in a high zoom ratio to provide a more accurate diagnosis.

In FIG. 9A, the arrangement of the instrument channel 26 allows different instrument to reach the first end 11a of the egg-shaped camera module. In this regard, the polypectomy snare 90 is extended into the instrument channel 26. The instrument channel 26 can also extend out of the instrument channel 26 to excise the polyp.

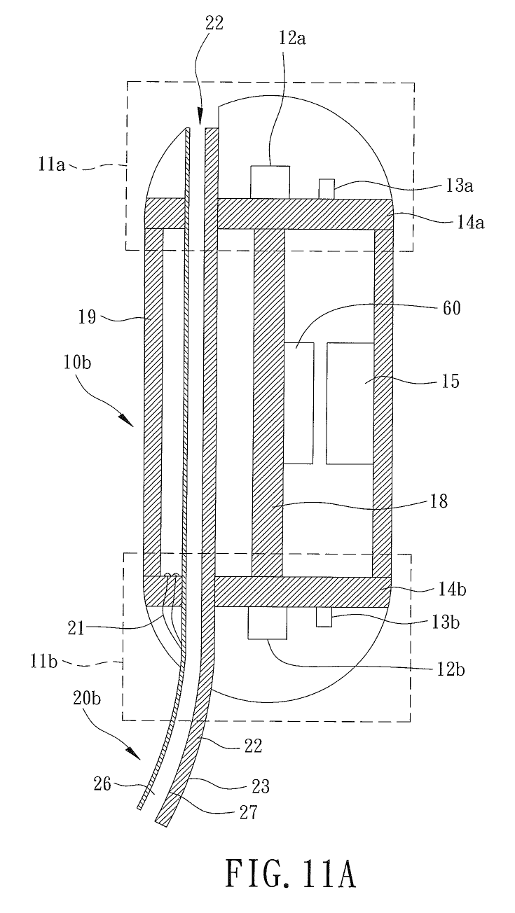

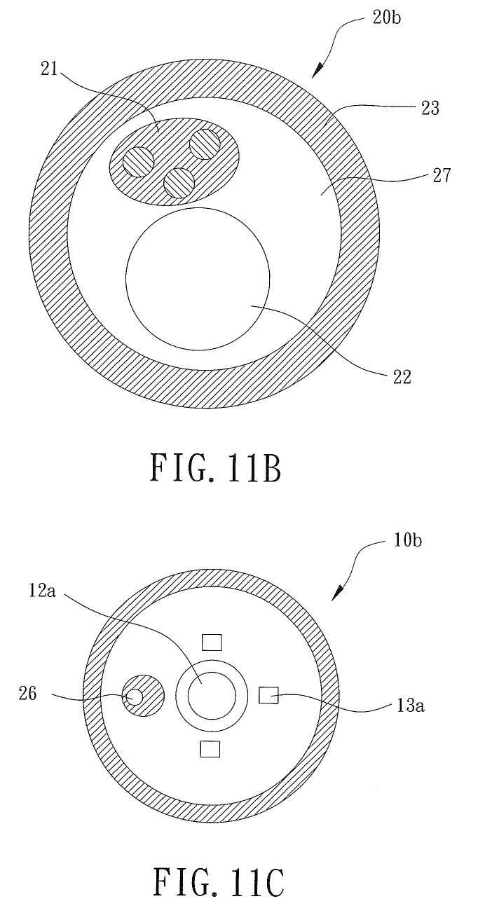

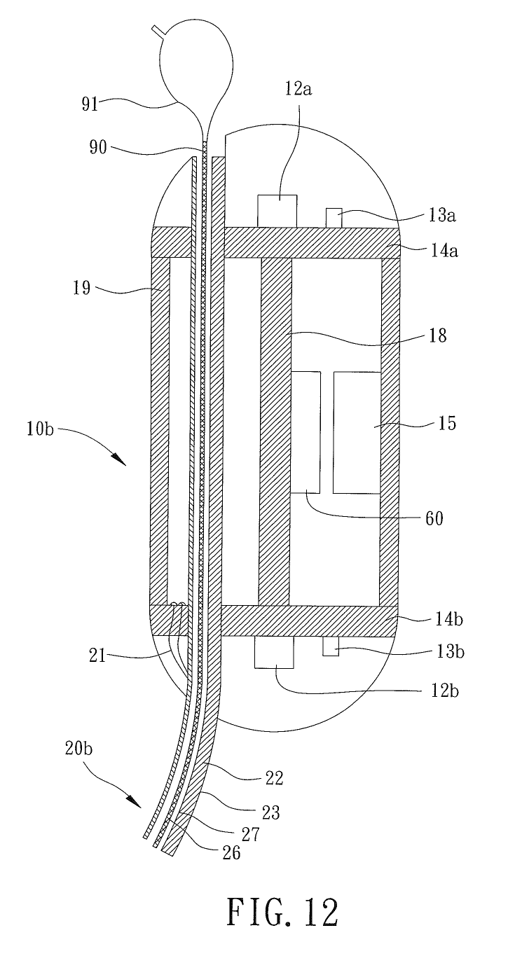

FIGS. 11A, 11B and 11C show an egg-shaped camera module of a colonoscope according to a fifth embodiment of the disclosure. In FIG. 11A, a cross sectional view of the egg-shaped camera module is shown. In FIG. 11B, a cross sectional view of an exposed part of a wiring unit of the camera module is shown. In FIG. 11C, a cross sectional view of the first end 11a of the camera module is shown. FIG. 12 is a cross sectional view of the egg-shaped camera module of FIGS. 11A-11C when the camera module is used in a surgery. In comparison with the fourth embodiment, the wiring unit 20a in the fifth embodiment does not include the instrument channel 26. Instead, the air tube 22 is used to replace the instrument channel. Thus, the air tube 22 allows the passage of air, liquid and instrument.

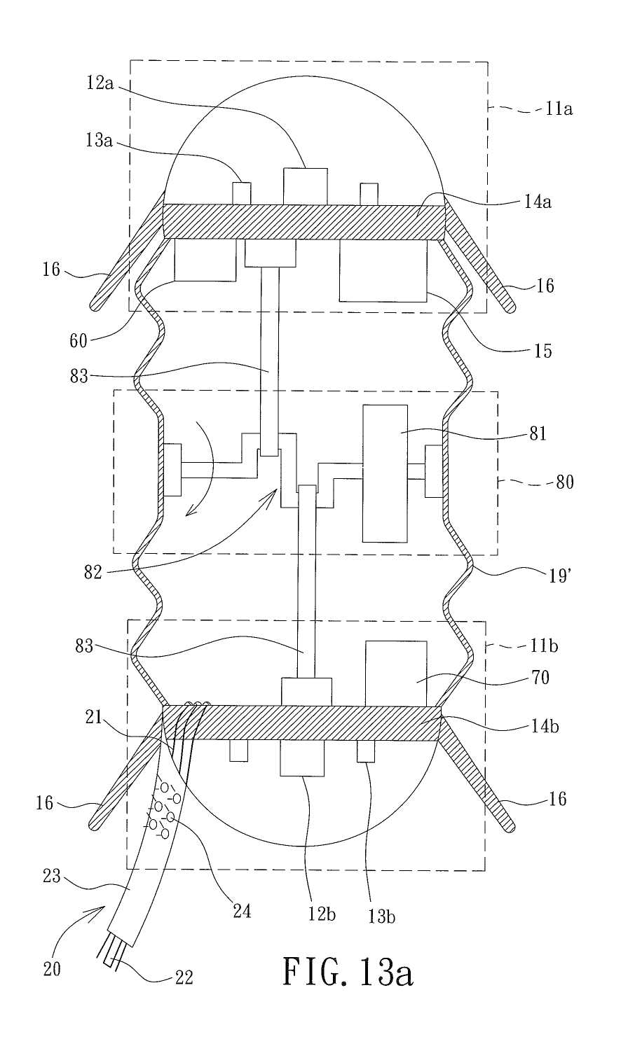

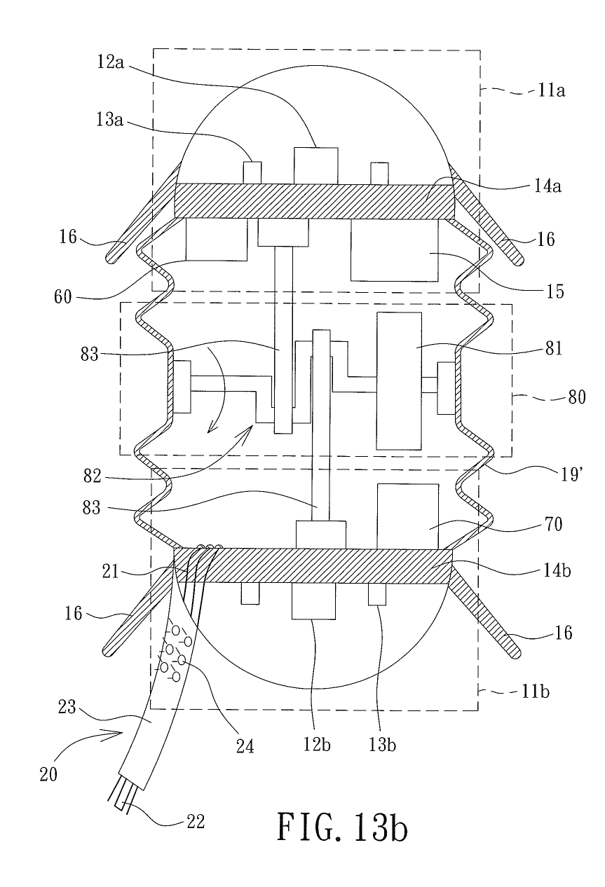

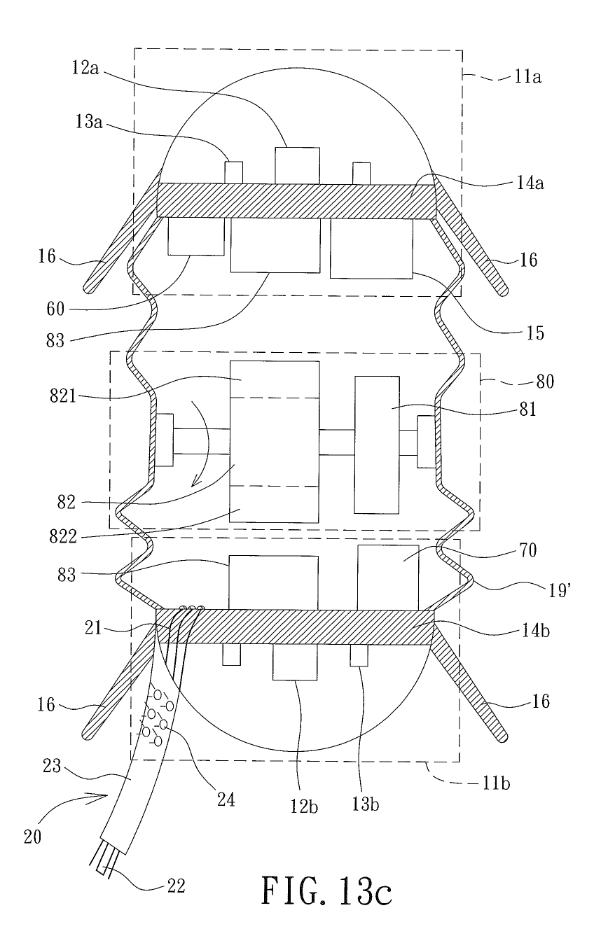

FIGS. 13A, 13B and 13C are cross sectional views of an egg-shaped camera module of a colonoscope according to a sixth embodiment of the disclosure. The egg-shaped camera module in this embodiment differs from that in the first embodiment in that the lateral wall 19′ of the egg-shaped casing 10c is made of a flexible material. Also, the egg-shaped camera module in this embodiment does not include the central circuit board 18. Therefore, the vibration motor 15, the control unit 60 and the angle detection unit 70 are mounted on the first circuit board 14a or the second circuit board 14b. In addition, the first circuit board 14a and the second circuit board 14b are connected to each other via a flexible lead wire. The egg-shaped camera module in the sixth embodiment is characterized in that the casing 10c includes a telescopic movement control unit 80 connected between the first end 11a and the second end 11b of the casing 10c. Also, the casing 10c includes the movement auxiliaries 16 on an outer surface thereof. Specifically, the telescopic movement control unit 80 may be a structure that allows the telescopic movement between the first end 11a and the second end 11b. The telescopic movement control unit 80 includes a drive motor 81, a telescopic driving member 82 and a telescopic driven member 83. The drive motor 81 can be controlled by the control unit 60 and is preferably mounted to an inner side of the lateral wall 19′. The telescopic driving member 82 is rotatably coupled with the drive motor 81. The telescopic driven member 83 is mounted to the first circuit board 14a (which is at the first end 11a) and the second circuit board 14b (which is at the second end 11b). In another approach, the drive motor 81 may be mounted to one of the first and second circuit boards 14a and 14b, and the telescopic driven member 83 may be mounted to another one of the first and second circuit boards 14a and 14b. In this arrangement, when the drive motor 81 drives the telescopic driving member 82, the telescopic driven member 83 will move away from the telescopic driving member 82 (as shown in FIG. 13A) or towards the telescopic driving member 82 (as shown in FIG. 13B). As a result, telescopic movement of the lateral wall 19′ is attained. Under the telescopic movement of the lateral wall 19′, the egg-shaped camera module can smoothly pass through the narrow area or bending area of the intestinal tract. The egg-shaped camera module can also move under the telescopic momentum only.

More specifically, the telescopic movement control unit 80 can include any structure that allows the repeated telescopic movement between the first end 11a and the second end 11b of the casing 10c. As shown in FIGS. 13A and 13B, the telescopic driving member 82 is in the form of a crankshaft, and the telescopic driven member 83 is in the form of two interconnected rods. One of the rods is connected to the first circuit board 14a, and the other one is connected to the second circuit board 14b. In another option as shown in FIG. 13C, the telescopic driving member 82 is in the form of a rotor having a first magnetic pole face 821 and a second magnetic pole face 822. The first and second magnetic pole faces have opposite magnetic poles and are arranged on an outer face of the rotor. In this regard, the telescopic driven member 83 may be in the form of two magnets facing the telescopic driving member 82. In this arrangement, the first magnetic pole face 821 may be N pole, the second magnetic pole face 822 may be S pole, the upper magnet 83 may be S pole, and the lower magnet 83 may be N pole. As such, the telescopic movement of the lateral wall 19′ may be attained based on the magnetically attractive and repulsive forces between the telescopic driving member 82 and the telescopic driven member 83. However, the rotor may include a plurality of first magnetic pole faces and a plurality of second magnetic pole faces that have opposite magnetic poles and are arranged on the outer face of the rotor in an alternating manner.

According to the structure of the egg-shaped camera module of the sixth embodiment, its forward movement control method can also control the camera module to move telescopically after the inclined angle of the camera module is detected. In this regard, the forward movement control method can slant the camera module in an inclined state where the first end 11a thereof is in a lower level than the second end 11b. Based on the inclined state of the camera module, the camera module can move forward under the telescopic momentum. Alternatively, the camera module can also move forward under the vibration force or the magnetic force (as mentioned previously) in addition to the telescopic force. Furthermore, since the casing 10c is provided with the movement auxiliaries 16, the movement auxiliaries 16 can abut against the intestinal wall during the telescopic movement of the camera module. As such, the forward movement of the camera module is facilitated.

The camera module of the disclosure can be used in a colonoscope to provide a colonoscopy which is nearly pain-free, has no blind spot, and prevents the intestinal perforation. The medical technology is significantly improved.

Although the disclosure has been described in detail with reference to its presently preferable embodiments, it will be understood by one of ordinary skill in the art that various modifications can be made without departing from the spirit and the scope of the disclosure, as set forth in the appended claims.

What is claimed is:

1. A colonoscope comprising:
a camera module comprising:
a casing having a first end and a second end, wherein the first and second ends are spaced from each other in an axial direction of the casing, and wherein the first end is made of a transparent material;
a first camera unit arranged at the first end of the casing to provide an illumination effect and to capture a first image in a first direction;
a vibration motor arranged in the casing and adapted to vibrate the casing; and
a control unit arranged in the casing and electrically connected to the first camera unit and the vibration motor, wherein the control unit controls the first camera unit to capture the first image and controls the transmission of the captured first image upon the reception of a command, and wherein the control unit controls the vibration of the vibration motor; and
a wiring unit fixed to the second end of the casing and comprising an outer sheath and an air tube, wherein the air tube is made of a soft material and is enveloped in the outer sheath; and
a power supply member disposed in the casing or the wiring unit and electrically connected to the control unit, wherein the power supply member provides power to the control unit,
wherein the casing or the wiring unit forms a vent which is in communication with the air tube of the wiring unit, wherein the air tube is adapted to convey an air, and wherein the vent is adapted to output the conveyed air to a colon.

2. The colonoscope as claimed in claim 1, further comprising a second camera unit arranged at the second end of the casing, wherein the second camera unit is adapted to capture a second image in a second direction substantially opposite to the first direction.

3. The colonoscope as claimed in claim 1, further comprising an angle detection unit electrically connected to the control unit, wherein the angle detection unit is adapted to detect an inclined angle of the casing with respect to a horizontal line, and wherein the control unit transmits a detected result of the inclined angle to an outer device.

4. The colonoscope as claimed in claim 3, wherein the angle detection unit is a microelectromechanical angle detection chip, a microelectromechanical gyroscope chip, a microelectromechanical dual-axis acceleration detection chip, a microelectromechanical tri-axis acceleration detection chip, a rolling switch, or a magnetic sensor.

5. The colonoscope as claimed in claim 1, further comprising at least one movement auxiliary arranged on an outer surface of the casing, wherein the at least one movement auxiliary is adapted to facilitate a movement of the camera module.

6. The colonoscope as claimed in claim 1, further comprising at least one internal magnetic member mounted to an inner face of the casing, wherein the at least one internal magnetic member is adapted to be magnetically attracted to an external magnetic member, so that a movement of the external magnetic member is able to cause a movement of the at least one internal magnetic member.

7. The colonoscope as claimed in claim 1, wherein the power supply member is in a form of a plurality of lead wires contained in the wiring unit.

8. The colonoscope as claimed in claim 1, further comprising a wireless transmission module connected to the control unit, wherein the wireless transmission module is adapted to transmit the captured first image to an outer device.

9. The colonoscope as claimed in claim 8, wherein the power supply member is in a form of a battery mounted in the casing, and wherein the wireless transmission module is adapted to receive and transmit the command to the control unit.

10. The colonoscope as claimed in claim 1, wherein the colonoscope further comprises a power-line signal transmission module or the wiring unit further comprises a signal line, wherein the power-line signal transmission module is connected to the control unit, where the power-line signal transmission module and the signal line are adapted to transmit the captured first image to an outer device.

11. The colonoscope as claimed in claim 1, wherein the casing has a length of 2.5-5.2 cm and a width of 1.5-2.5 cm.

12. The colonoscope as claimed in claim 1, wherein the wiring unit or the air tube further comprises an instrument channel provided for insertion of an instrument, wherein the instrument channel has an outlet at the first end of the casing, and wherein the instrument is adapted to extend into the instrument channel and extend out of the outlet for performing a surgery.

13. The colonoscope as claimed in claim 1, further comprising a telescopic movement control unit connected between the first end and the second end of the casing, wherein the casing has a lateral wall made of a flexible material.

14. The colonoscope as claimed in claim 13, wherein the casing comprises at least one movement auxiliary on an outer surface thereof.

15. The colonoscope as claimed in claim 13, wherein the telescopic movement control unit comprises a drive motor, a telescopic driving member and a telescopic driven member, wherein the drive motor is mounted to an inner side of the lateral wall, wherein the telescopic driving member is rotatably coupled with the drive motor, and wherein the telescopic driven member is mounted to at least one of the first and second ends.

16. A forward movement control method of a camera module of a colonoscope, wherein the colonoscope further comprises a wiring unit, wherein the camera module comprises a vibration motor, a first end, a second end and a first camera unit, wherein the vibration motor is adapted to vibrate the casing, wherein the wiring unit is adapted to transmit power, wherein the first and second ends are spaced from each other in an axial direction of the casing, wherein the first camera unit is located at the first end of the casing, wherein the method comprises:
vibrating the camera module; and
slanting the camera module in an inclined state where the first end of the casing is in a lower level than the second end during the vibration of the camera module.

17. The forward movement control method of the camera module of the colonoscope as claimed in claim 16, further comprising:
providing a pulling force to regulate a movement speed of the camera module and to adjust a movement direction of the camera module.

18. The forward movement control method of the camera module of the colonoscope as claimed in claim 17, wherein providing the pulling force comprises:
pulling the wiring unit to generate the pulling force.

19. The forward movement control method of the camera module of the colonoscope as claimed in claim 16, further comprising:
detecting an inclined angle of the casing with respect to a horizontal line; and
transmitting a detected result of the inclined angle to an outer device.

20. A forward movement control method of a camera module of a colonoscope, wherein the colonoscope further comprises a wiring unit, wherein the camera module comprises a vibration motor and an internal magnetic member, wherein the vibration motor is adapted to vibrate the camera module, wherein the wiring unit is adapted to transmit power, wherein the method comprises:
vibrating the camera module;
providing an external magnetic member that is magnetically attracted to the internal magnetic member; and
moving the external magnetic member to cause movement of the internal magnetic member via a magnetic force between the internal and external magnetic members during the vibration of the camera module, so as to facilitate a movement of the camera module.

21. The forward movement control method of the camera module of the colonoscope as claimed in claim 20, further comprising:
providing a pulling force to regulate a movement speed of the camera module and to adjust a movement direction of the camera module.

22. The forward movement control method of the camera module of the colonoscope as claimed in claim 21, wherein providing the pulling force comprises:
pulling the wiring unit to generate the pulling force.

23. The forward movement control method of the camera module of the colonoscope as claimed in claim 20, further comprising:
detecting an inclined angle of the casing with respect to a horizontal line; and
transmitting a detected result of the inclined angle to an outer device.

24. A forward movement control method of a camera module of a colonoscope, wherein the colonoscope further comprises a wiring unit, wherein the camera module comprises a first end, a second end, a first camera unit and a telescopic movement control unit, wherein the wiring unit is adapted to transmit power, wherein the first and second ends are spaced from each other in an axial direction of the camera module, wherein the first camera unit is located at the first end of the casing, wherein the telescopic movement control unit is adapted to control the camera module to move telescopically along the axial direction of the camera module, wherein the method comprises:
moving the camera module telescopically; and
slanting the camera module in an inclined state where the first end of the camera module is in a lower level than the second end during the telescopic movement of the camera module.

25. The forward movement control method of the camera module of the colonoscope as claimed in claim 24, wherein the camera module further comprises at least one movement auxiliary on an outer surface thereof, wherein the method further comprises:
abutting the at least one movement auxiliary against an intestinal wall of a colon during the telescopic movement of the camera module.
